(19) United States
(12) Patent Application Publication
(10) Pub. No.: US 2018/0020968 A1
(43) Pub. Date: Jan. 25, 2018

(54) SYSTEM, DEVICE, METHOD AND COMPUTER PROGRAM FOR PROVIDING A HEALTH ADVICE TO A SUBJECT

(71) Applicant: KONINKLIJKE PHILIPS N.V., EINDHOVEN (NL)

(72) Inventors: Johan Partomo DJAJADININGRAT, Utrecht (NL); Hanneke HOVELS, Eindhoven (NL); Marie PEREZ, Eindhoven (NL); Wing Lam LUI, Den Haag (NL)

(21) Appl. No.: 15/536,484

(22) PCT Filed: Dec. 9, 2015
(86) PCT No.: PCT/EP2015/079047

§ 371 Date: Jun. 15, 2017

(30) Foreign Application Priority Data
Dec. 18, 2014 (EP) 14198968.1

(51) Int. Cl. A61B 5/00; G09B 5/02; G06F 19/00; G09B 5/00; G09B 5/06; G09B 19/00; G09B 5/04

(52) U.S. Cl. CPC A61B 5/486; A61B 5/7405; A61B 5/742; A61B 5/7455; G09B 19/0092; G09B 5/02; G09B 5/04; G09B 5/00; G09B 5/06; G09B 19/00; G06F 19/345; G06F 19/3406; G06F 19/3431

(57) ABSTRACT

The present invention relates to a system (100), a device, a method (200), and a computer program for providing a health advice to a subject. It is proposed to modify a digital coaching advice depending on trends of a physiological observable (e.g., weight, Body Mass Index, etc.). A digital coach varies the wording of his/her advice depending on the risk categorization. It is proposed to analyze physiological measurements for deviation from the mean and rate of change and to translate these basic statistics into a concept of urgency/risk to structurally vary the wording or appearance of a digital coach. Thus, a digital coach as proposed herein may look, act and talk differently depending on the urgency/risk of the users. Coaches may vary in language and appearance from gentle, via hard-but-fair, to “drill sergeant”.

### FIELD OF THE INVENTION

The invention relates to a system for providing a health advice to a subject, a device for providing a health advice to a subject, a method for providing a health advice to a subject, and a computer program for providing a health advice to a subject.

### BACKGROUND OF THE INVENTION

Unhealthy lifestyle is one of the root causes of chronic conditions. In order to improve their conditions, patients are often required to change one or more lifestyle habits and/or behaviors. Studies have shown that unhealthy habits lead to more illness and hospitalization. More importantly, studies demonstrated that amending unhealthy habits is often not straightforward and maintaining changes in behavior over time is challenging for the patient. Considering the automatic, powerful, context-driven nature of habits, habits are hard to change. Means to help a patient are proposed, but they generally include health coaches (human or automatic).

Current automatic coaches typically take into account two binary (yes/no) variables into consideration: 1) actual value within acceptable range and 2) change from non-acceptable to acceptable or vice versa.

U.S. Pat. No. 6,609,068 B2 discloses a medical breath component analyzer which maintains a data-base profile of a patient over time. The apparatus may be used chronically by a patient so that a baseline status for that patient may be determined. Acute variations from the baseline are identified as clinically significant. The acquired data can be reported to the patient using the device at home and transmitted electronically to a physician or health care provider. The method and apparatus helps a patient modify health related behaviors, particularly weight loss for diabetic patients. A breath component and information on the psychological or emotional state of the patient are correlated, and information is provided to the patient based on the correlation between the breath component and the patient's emotional state. Other physiologic parameter may also be measured, such as a blood component, temperature, cardiovascular condition or pulse rate, a urine component, a physical activity sensor, weight, or body fat composition sensor. The parameters and the information on the patient's emotional state may be correlated through a computer system. Correlation may comprise selecting a response likely to re-enforce positive behavioral change in the patient. Preferably, remote sources of information may also be accessed, as, for example, through a communications connection or the Internet. Information may be provided directly from the apparatus, or by contact through a physician, health-care provider or support group.

The problem with known automatic coaches is that it enables only a yes and no decision; the system does not start to react to a problem until the actual measured value is outside the acceptable range, i.e., past a predefined threshold). As long as the measured value stays within the acceptable range, the feedback remains positive, even though the trend or the position within the acceptable range may already suggest that things are likely to go wrong. Further, automatic coaches tend to be very repetitive, thereby potentially leading to the subject not paying close attention to the automatic coach anymore.

US 2006/0015016 A1 describes a system and methodology for developing interactive weight control and maintenance programs. A weight control program is developed during a visit to a treating physician or expert based on medical data, medical historical data, and patient profile data. The data and program is then stored in a host computer and relevant portions are transferred to a weight measuring and feedback unit including a scale and a processing unit having audio and/or visual and/or tactile output devices for patient feedback, which is taken to the patient's home. The patient then weighs himself/herself on a periodic basis and the apparatus issues recommendations or other messages for improved compliance.

### SUMMARY OF THE INVENTION

It is an object of the present invention to provide an improved system, device, method, and computer program for providing a health advice to a subject, which overcome the disadvantages of the prior art.

In a first aspect of the invention, there is provided a system for providing a health advice to a subject, the system comprising: a health level determining unit that is configured to determine a health level of the subject for a first time, wherein the health level is based on a comparison of a physiological observable of the subject at the first time to an target physiological observable of the subject; a health advice providing unit that is configured to provide a health advice to the subject based on the physiological observable of the subject at the first time and on a rate of change of the physiological observable of the subject over time; and an urgency assigning unit that is configured to assign a degree of urgency to the health advice based on the health level of the subject at the first time and on a rate of change of the physiological observable of the subject over time.

In order to solve the aforementioned problem, an algorithm which takes into account the difference between the measured value and the target value as well as the rate of change is proposed. The algorithm is configured to output a “level of urgency” based on the rate of change between two measurements. In an embodiment, the algorithm may further consult a look-up table in which each piece of advice has been (pre)worded for different levels of urgency, so that the advice can be offered in a way which is appropriate to the measured condition of the user. Put differently, it is proposed to analyze physiological measurements for deviation from the mean and rate of change and to translate these basic statistics into a concept of urgency/risk to structurally vary the wording or appearance of a digital coach. Thus, a digital coach as proposed herein may look, act and talk differently depending on the urgency/risk of the users.

It is noted that the “first time” corresponds to the actual time of measurement of the physiological observable. Typically, the “first time” will be close in time to a time when the health advice is provided to the user. This is because providing the health advice based on outdated measurement data will only provide health advice on what the user should have done at the time of measurement, which might not be appropriate at the time of providing the advice to the user. Nonetheless, it is conceivable that the “first time” differs from the time or providing the health advice for instance for the case of ex post facto health behavior analyses, where the user may be confronted with different user interface appearances all being determined based on the same “first time”. Such an ex post facto analysis may thus serve to “calibrate” the system to a respective user by looking for the optimal user interface appearance.

It is further noted that the health advice may in principle also be provided based on the health level of the subject at the first time and on a rate of change of the health level of the subject over time. Referring to the physiological observable rather than to the health level is however better suited for cases where the granularity of the health levels is very low (i.e., only few health levels) so that a determined health level might not change even though the respective physiological observable is rapidly moving towards the health level edges.

The term “observable” generally refers to the result when measuring a specific property. Thus, a physiological observable corresponds to the result when measuring a specific physiological property, such as, e.g., weight, blood pressure, etc.

The term “rate of change” is generally used to describe how a measurement outcome changes with respect to another parameter. For instance, when considering a physiological observable's time evolution, if the measurement outcome strongly changes in a small time interval, the rate of change is large. If the measurement weakly changes in a small time interval, the rate of change is small. On option to calculate the rate of change of a physiological observable Φ between two times t and t+dt would be to consider the ratio Φ(t+dt)−Φ(t)/dt. Other ways to specify the rate of change are known to the person skilled in the art.

In an embodiment, the system may comprise a first database for storing the target value of one or more physiological parameters for a patient. The first database further comprises data which are in the vicinity of the target value, as well as the definition of minimum and maximum values, indicating the outer bounds of the acceptable, healthy range. The system may comprise means to measure said one or more physiological parameter over time. The system may further comprise means for calculating the rate of change (slope) over time. The system may further comprise a second database, herein also referred to as a feedback urgency database, comprising a table defining the urgency of the feedback based on the difference between the measured and target value as well as the rate of change of the signal. Based on detected urgency, an appropriate advice is picked from a look-up table and provided to the patient/alternatively an appropriate “tone of voice” or “image” is chosen for adequately instruct the patient.

Means are proposed to assess, based on measured physiological parameter(s), what is the best advice (coaching) to be presented to a subject. The system is capable of discriminating between different levels of “non-ideal values”.

Specifically, an algorithm is proposed which takes into account the difference between the actual, measured value and the ideal value as well as the rate of change. The combination of these two parameters results in an urgency rating. The system may comprise a look-up table in which each piece of advice has been worded for different levels of urgency, so that the advice can be offered in a tone of voice which is appropriate to the measured condition of the user. This means that the system can vary its feedback from small, gentle nudges when the user wavers a little off-target to very urgent warnings when the measured parameter changes drastically. The system's feedback is thus less binary and black and white, and more analogue and granular.

In weight management, for example, a sudden change of slope may signal an early phase of “binge eating” which requires the digital coach to react with a highly concerned, urgent response, even though the measured weight itself may not yet be a cause for concern. Also, a weight measurement on the limits of the acceptable range will result in far more intense feedback than a measurement near the target value, even though both are within the acceptable range.

The system's feedback is thus less black and white and less repetitive, and more granular and appropriate to the user's condition, resulting in digital coaching which feels more human.

The ideal target value need not be a constant: it may increase (e.g. weight during pregnancy), it may fluctuate according to a known pattern (e.g. blood pressure during pregnancy) or it may decrease (e.g. weight management of the mother after birth). Therefore over time, the upper and lower limit of the acceptable range and the sub-ranges are preferably changed with the target value.

In an embodiment, a “tone of voice” of the coaching advice is dependent on two variables: the difference between actual value and target value and the rate of change of the measurement. Thus, the system may generally provide for “tones of voice” of the health advice which vary with urgency. Further, a difference between an actual measurement of a physiological observable and a target value of said physiological observable (e.g., weight) results in a structured variation of the “tone of voice” rather than simply adding the variation in “tone of voice” to make the digital coach less repetitive. Further, a change in the rate of change of the actual measurement results in a structured variation of the “tone of voice”. Specifically, in an embodiment, it is proposed that the difference between actual and target as well as the rate of change jointly result in an urgency rating. This may be done as follows: divide both the deviation and the slope into bands (e.g., low, medium and high) and create a table with resulting urgency. The simplest ‘algorithm’ would simply be to add the deviation band to the slope band.

In an embodiment, an avatar may be used as the “health coach”, wherein a representation of said avatar in light of the detected level of urgency is modified.

The present invention proposes to modify a digital coaching advice depending on trends. This is based on the concept of understanding urgent and less urgent trends in order to structurally vary the wording of the coaching advice. The present invention facilitates communication between users and a digital coach rather than communication between users and medical professionals. In accordance with an embodiment, a digital coach varies the wording of his/her advice depending on the risk categorization. Put differently, it is proposed to analyze physiological measurements for deviation from the mean and rate of change and to translate these basic statistics into a concept of urgency/risk to structurally vary the wording or appearance of a digital coach. Thus, a digital coach as proposed herein may look, act and talk differently depending on the urgency/risk of the users. Coaches may vary in language and appearance from gentle, via hard-but-fair, to “drill sergeant”.

In an embodiment, the effectiveness of wording and appearance of a digital coach is analyzed in relation to urgency measurements to optimize the digital coach for not being too soft or too harsh on the subject.

In an embodiment, the system further comprises a user interface, wherein the system is configured provide an appearance of the user interface based the degree of urgency. In an embodiment, the system is configured to provide a feedback to the user based on the assigned degree of urgency, wherein the feedback to the user is at least one of a visual feedback, an auditory feedback, a haptic feedback, and/or an olfactory feedback.

In an embodiment, the system is configured to provide at least one of a tone of voice and/or an instructor image based on the assigned degree of urgency. Accordingly, the user receives prompt feedback where the deviation from the mean and the rate of change are used to signal to the user the level of urgency in an immediately understandable manner (e.g., by picturing a drill instructor for urgent messages). In other words, the system is configured to provide (1) a tone of voice, (2), an instructor image or (3) both of a tone of voice and an instructor image. In an embodiment, the system is configured to provide more than one tone of voice and/or more than one instructor image.

In an embodiment, the at least one of a visual feedback, an auditory feedback, a haptic feedback, and/or an olfactory feedback based on the assigned degree of urgency are selected from a memory unit. In an embodiment, the memory unit comprises at least one of a look-up table and/or a parameterized language database.

In a further embodiment, the at least one of a tone of voice and/or an instructor image based on the assigned degree of urgency are selected from at least one of a look-up table and/or a parameterized language database. When using parameterized language, for example, a sentence could be worded in different ways using words which have similar meaning but increasing strength, such as “could”→“should”→“must” or “unwise”→“irresponsible”→“stupid”. In an embodiment, the system is configured to provide more than one tone of voice and/or more than one instructor image.

In a further embodiment, the system further comprises a first database for storing the target physiological observable of the subject. By storing the target physiological variable, the system can promptly determine the user's health level without the need to receive a respective value first (e.g., via input receiving means such as a user interface).

In a further embodiment, the first database is further configured to store a healthy range of the physiological observable of the subject and a parameter corresponding to a total number of health levels. Having stored the healthy range and total number of health levels, the respective sub-ranges between subsequent health levels could be predetermined in order to save computation time when determining the health level for a given time.

In a further embodiment, the system further comprises a physiological observable providing unit that is configured to provide the physiological observable of the subject at the first time. In a further embodiment, the physiological observable providing unit comprises an input unit that is configured to receive the physiological observable of the subject at the first time. In a further embodiment, the physiological observable providing unit comprises a sensor that is configured to measure the physiological observable of the subject at the first time. In an embodiment, the physiological observable providing unit is configured to provide the physiological observable of the subject at the first time to the health level determining unit. In an embodiment, the physiological observable providing unit is configured to provide the physiological observable of the subject at the first time to the health advice providing unit.

In a further embodiment, the system further comprises a rate of change providing unit that is configured to calculate the rate of change of the physiological observable of the subject over time. By being able to calculate the rate of change of the health level, the system can promptly determine the degree of urgency without the need to receive a respective value for the rate of change first (e.g., via input receiving means such as a user interface).

In a further embodiment, the system further comprises a rate of change providing unit that is configured to receive the rate of change of the physiological observable of the subject over time via a respective input port.

In a further embodiment, the system further comprises a second database that is configured to store a table defining the degree of urgency depending on the health level of the subject at a given time and on the rate of change of the physiological observable of the subject over time. The information comprised in such a table thereby mimics a human coach who would potentially adapt his/her tone of voice or choice of wording in order to avoid the user from paying less attention or being less motivated to stay within (or reach) the healthy range.

In a further embodiment, the value of the target physiological observable of the subject is dependent on time. The ideal, target value need not be a constant. It may increase (e.g. weight during pregnancy), it may fluctuate according to a known pattern (e.g. blood pressure during pregnancy) or it may decrease (e.g. weight management of the mother after birth). Therefore over time, the upper and lower limit of the acceptable range and the sub-ranges need to change with the target value.

In a further embodiment, the system further comprises an effectiveness analyzing unit that is configured to correlate the assigned degree of urgency over time with the determined health level over time, wherein the effectiveness analyzing unit is further configured to provide an effectiveness rating based on the correlation. By analyzing the effectiveness of wording and appearance of a digital coach in relation to urgency measurements, the digital coach may be optimized to not be too soft or too harsh on the subject. For instance, if an analysis yields the result that a chosen mapping of degree of urgency and appearance of a user interface has a high impact on user behavior (e.g., user reacts quickly to health advice and physiological observable quickly returns to target value), the corresponding respective effectiveness rating would also be high. In contrast, if an analysis yields the result that a chosen mapping of degree of urgency and appearance of a user interface has a low impact on user behavior (e.g., user behavior is unaffected by the digital coach), the corresponding respective effectiveness rating would be low.

In a further embodiment, the system further comprises a display unit that is configured to display the health advice taking into account the assigned degree of urgency. In a further embodiment, the system further comprises a speaker unit that is configured to provide the health advice as an acoustic sound taking into account the assigned degree of urgency.

A further embodiment of the system forms device for providing a health advice to a subject. The device comprises a system for providing health advice, as described above in accordance with the first aspect of the invention;

a degree of urgency providing unit that is configured to provide the degree of urgency corresponding to the health advice; and

a user interface that is configured to provide the health advice to the subject, wherein the user interface is configured to vary an appearance of the health advice depending on the degree of urgency.

In a second aspect, there is provided a device for providing a health advice to a subject, the device comprising: a health advice providing unit that is configured to provide a health advice based on a physiological observable of the subject at a first time and on a rate of change of the physiological observable of the subject over time; and a degree of urgency providing unit that is configured to provide a degree of urgency corresponding to the health advice based on the health level of the subject at the first time and on a rate of change of the physiological observable of the subject over time; and a user interface that is configured to provide the health advice to the subject, wherein the user interface is configured to vary an appearance of the health advice depending on the degree of urgency.

In an embodiment of the device for providing a health advice to a subject according to the embodiment of the system of the first aspect, and in an embodiment of the device for providing a health advice to a subject according to the second aspect, the user interface that is configured to provide the health advice to the subject by means of at least one of a visual feedback, an auditory feedback, a haptic feedback, and/or an olfactory feedback; the user interface is further configured to vary the at least one of a visual feedback, an auditory feedback, a haptic feedback, and/or an olfactory feedback depending on the degree of urgency.

In a further aspect, there is provided a method for providing a health advice to a subject, the method comprising the steps of: determining a health level of the subject for a first time, wherein the health level is based on a comparison of a physiological observable of the subject at the first time to a target physiological observable of the subject; providing a health advice based on the physiological observable of the subject at the first time and on a rate of change of the physiological observable of the subject over time; and assigning a degree of urgency to the health advice based on the health level of the subject at the first time and on a rate of change of the physiological observable of the subject over time.

The step of assigning a degree of urgency may in principle be performed before or after the step of providing a health advice. User feedback is provided based on the health advice itself (e.g., “reduce salt intake”) and based on the degree of urgency (e.g., low or high alert), but the health advice as such does not necessarily need to be present when determining the degree of urgency. That is, in an embodiment, it is conceivable to determine the degree of urgency and only then retrieve the respective health advice.

In a further aspect, there is provided a computer program for providing a health advice to a subject, the computer program comprising program code means for causing the system for providing a health advice to a subject to carry out the steps of the method for providing a health advice to a subject, when the computer program is run on a computer controlling the system for providing a health advice to a subject.

It shall be understood that the system for providing a health advice to a subject of claim 1, the device for providing a health advice to a subject of claim 12, the method for providing a health advice to a subject of claim 14 and the computer program for providing a health advice to a subject of claim 15 have similar and/or identical preferred embodiments as defined in the dependent claims.

It shall be understood that a preferred embodiment of the invention can also be any combination of the dependent claims or above embodiments with the respective independent claim.

These and other aspects of the invention will be apparent from and elucidated with reference to the embodiments described hereinafter.

### BRIEF DESCRIPTION OF THE DRAWINGS

In the following drawings:

FIG. 1 shows schematically and exemplarily an embodiment of a system for providing a health advice to a subject,

FIG. 2 shows schematically and exemplarily an embodiment of a method for providing a health advice to a subject,

FIG. 3 illustrates a conventional approach for providing feedback on a user's health behavior, and

FIG. 4 shows an approach for providing feedback on a user's health behavior in accordance with the present invention.

### DETAILED DESCRIPTION OF EMBODIMENTS

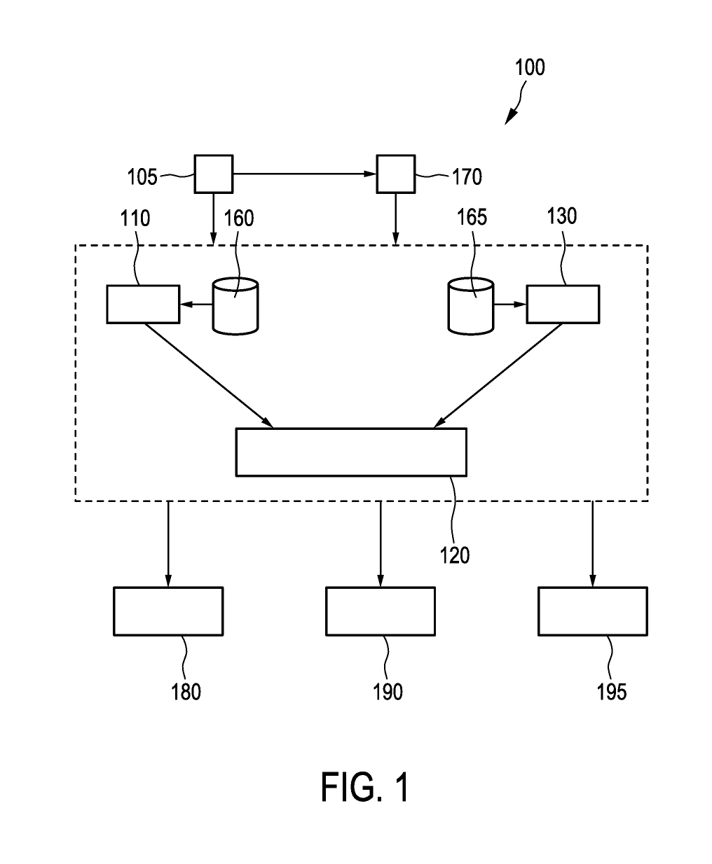

FIG. 1 shows schematically and exemplarily an embodiment of a system 100 for providing a health advice to a subject. System 100 comprises health level determining unit 110 that is configured to determine a health level of the subject for a first time, wherein the health level is based on a comparison of a physiological observable of the subject at the first time to a target physiological observable of the subject. System 100 further comprises health advice providing unit 120 that is configured to provide a health advice to the subject based on the physiological observable of the subject at the first time and on a rate of change of the physiological observable of the subject over time. System 100 further comprises an urgency assigning unit 130 that is configured to assign a degree of urgency to the health advice based on the health level of the subject at the first time and on a rate of change of the physiological observable of the subject over time.

Optional physiological observable providing unit 105 is configured to provide the physiological observable of the subject at the first time. Physiological observable providing unit 105 may comprise an input unit that is configured to receive the physiological observable of the subject at the first time. Additionally and/or alternatively, physiological observable providing unit 105 may comprise a sensor that is configured to measure the physiological observable of the subject at the first time.

Optional rate of change providing unit 170 is configured to calculate the rate of change of the physiological observable of the subject over time.

Optional first database 160 is provided for storing the target physiological observable of the subject. First database 160 may be further configured to store a healthy range of the physiological observable of the subject and a parameter corresponding to the total number of health levels. Optional second database 165, which herein is also referred to as the feedback urgency database, is configured to store a table defining the degree of urgency depending on the health level of the subject at a given time and on the rate of change of the physiological observable of the subject over time.

Optional effectiveness analyzing unit 180 is configured to correlate the assigned degree of urgency over time with the determined health level over time. Effectiveness analyzing unit 180 is further configured to provide an effectiveness rating based on the correlation.

Optional display unit 190 is configured to display the health advice taking into account the assigned degree of urgency. Optional speaker unit 195 is configured to provide the health advice as an acoustic sound taking into account the assigned degree of urgency.

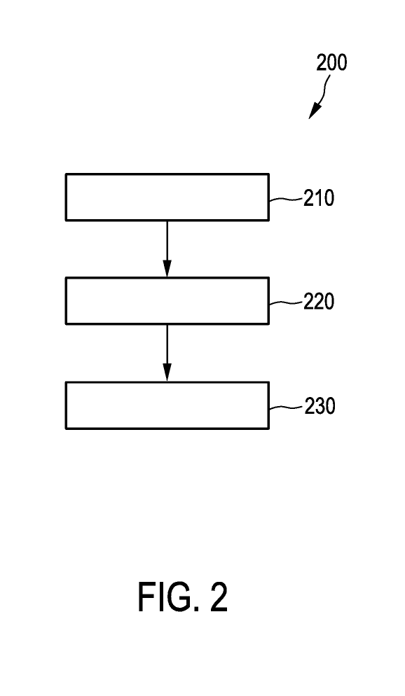

FIG. 2 shows schematically and exemplarily an embodiment of a method 200 for providing a health advice to a subject. In a step 210, a health level of the subject for a first time is determined. The health level is based on a comparison of a physiological observable of the subject at the first time to a target physiological observable of the subject. In a step 220, a health advice is provided based on the physiological observable of the subject at the first time and on a rate of change of the physiological observable of the subject over time. In a step 230, a degree of urgency is assigned to the health advice based on the health level of the subject at the first time and on a rate of change of the physiological observable of the subject over time.

The step of assigning a degree of urgency may in principle be performed before or after the step of providing a health advice. User feedback is provided based on the health advice itself (e.g., “reduce salt intake”) and based on the degree of urgency (e.g., low or high alert), but the health advice as such does not necessarily need to be present when determining the degree of urgency. That is, in an embodiment, it is conceivable to determine the degree of urgency and only then retrieve the respective health advice.

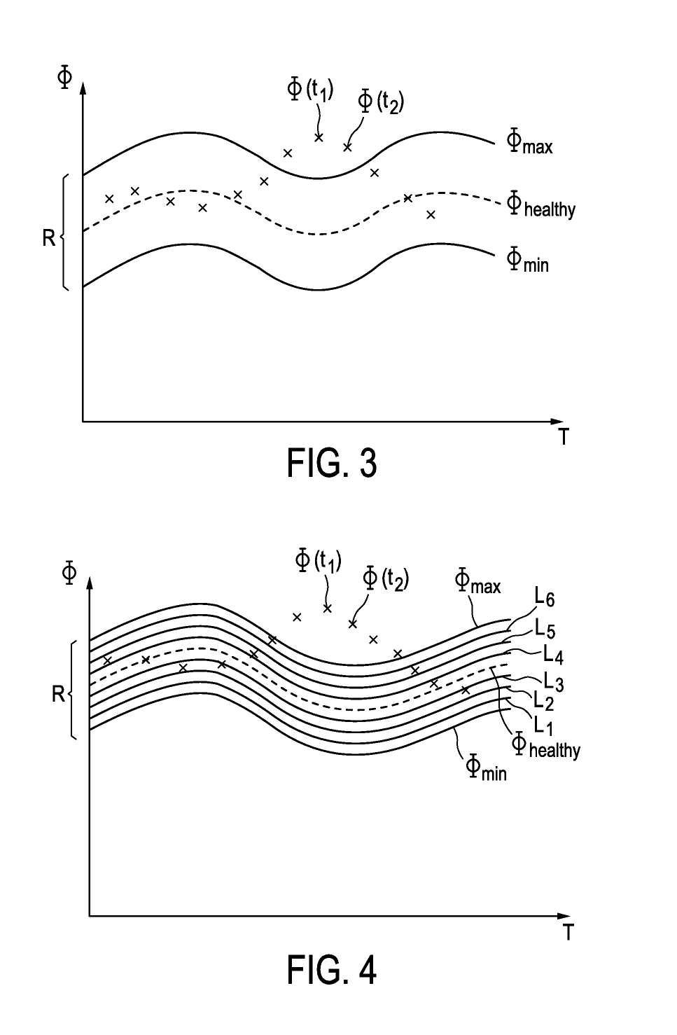

FIG. 3 illustrates a conventional approach for providing feedback on a user's health behavior. According to the simple model illustrated in FIG. 3, measurements Φ(t₁), Φ(t₂) of a physiological observable Φ (e.g., weight, blood pressure, calorie intake, physical activity, etc.) at times t₁, t₂, preformed over time T fall inside or outside an acceptable range R around a target value Φ_{healthy} and do or do not exit/enter the acceptable range R, leading to four possible levels of feedback. In the example shown, even if a measurement already shows the risk of going outside the acceptable range (e.g., based on the slope of the curve), a conventional algorithm's feedback remains positive as long as the measured observable remains within the accepted range between Φₘᵢₙ and Φₘₐₓ.

This leads to a 2×2 matrix and four types of feedback. Example feedback messages for the conventional approach illustrated in FIG. 3 are given in the subsequent table:

| | no entering/exit of | |
|---|---|---|
| | acceptable range | entering/exit of acceptable range |
| inside | e.g. “Well done, you have | e.g. “What an achievement, |
| acceptable | maintained your target | you have reached your |
| range | weight for the last | target weight!” |
| | 17 days.” | |
| outside | e.g. “Your weight | e.g. “Careful, you have gained |
| acceptable | continues to be above | too much weight.” |
| range | what is considered | |
| | healthy.” | |

FIG. 4 shows an approach for providing feedback on a user's health behavior in accordance with the present invention. Here, an improved model is shown in which the acceptable range R has been subdivided into different zones with corresponding health levels L₁, . . . , L₆ and the rate of change is also considered. By taking the trend (or slope) of the curve into consideration, a measurement associated with a large rate of change warrants a more stern and urgent feedback, even if its actual value is still well within the acceptable range. In addition, measurements on the outer regions of the acceptable range likewise warrant a more stern and urgent feedback than measurements that are closer to the target line Φ_{healthy}.

The urgency level of feedback messages is thus made dependent on both the deviation from the target value and the rate of change in unhealthy direction. The urgency levels shown are intended to illustrate the working principle and are not to be taken as absolutes.

As a first example, the time evolution of a subject's Body Mass Index (BMI) is considered. The healthy range is between 20 to 25 kg/m². The following three cases are compared:

1. Subject is consistently at 24.5 kg/m². This is on the outer edge of the acceptable range but the rate of change is zero.
2. Subject is at 24.5 kg/m², but subject's BMI is increasing. Not only is the subject on the outer edge of the acceptable range, but the subject is also expected to leave the healthy range in the very near future.
3. Subject is at 22.5 kg/m² but subject's BMI is shooting up. Subject is right in the middle of the safe zone but its weight is changing rapidly. This could be a sign of ‘yo-yo-ing’, binge eating or another eating disorder.

These three cases warrant a different “tone of voice”. In the first case, there is room for improvement in the subject's lifestyle but at least everything is under control so there is no need for an alarming response: some gentle nudging will do. In the second case, though the deviation between actual and target BMI is exactly the same as in case 1, this is far more serious as the subject is heading for unhealthy values: the digital coach should advice with an urgent tone of voice. In the third case, even though the absolute BMI value is in line with the target there is something seriously wrong with the subject: subject's weight is changing dramatically and again the digital coach should help with urgency to address a potential eating disorder.

As a further example, example feedback messages which are modified in dependence on the deviation from the target value and on the rate of change are given in the subsequent table:

| | | Slope of graph in unhealthy direction (rate of change) | | |
|---|---|---|---|---|
| | | low | medium | high |
| deviation | small | 0 - current lifestyle | 1 - user is about to | 2 - careful: user is |
| from | | is fine | lose optimal | losing best level |
| target | | | lifestyle | habits! |
| value | medium | 1 - user is doing | 2 - there is room for | 3 - careful: user is |
| | | well, but there is | improvement and | rapidly losing good |
| | | room for | user is straying | habits! |
| | | improvement | from the right path | |
| | large | 2 - user's lifestyle | 3 - there is | 4 - be very careful: |
| | | is stable but there is | considerable room | user's condition is |
| | | considerable room | for improvement | suboptimal and |
| | | for improvement | and the user is | his/her condition is |
| | | | straying further | rapidly becoming |
| | | | from the right path | worse! |
| | extra large | 3 - user is on the | 4 - be very careful! | 4 - alert: user is on |
| | | edge of what is | user is on the edge | the edge of what is |
| | | healthy) | of what is healthy | healthy and his/her |
| | | | and his/her | condition is rapidly |
| | | | condition is | becoming worse! |
| | | | becoming worse | |
| | out of | 5 - high alert: user's | 5 - high alert: user's | 5 - high alert: user's |
| | healthy | condition is | condition is | condition is |
| | range | unhealthy! | unhealthy and | unhealthy and |
| | | | straying further! | rapidly |
| | | | | deteriorating |
| | | | | further! |

The different levels, for a given subject, may be organized as follows:

$$L = ⌊ \frac{\vert {Φ}_{measured} - {Φ}_{healthy} \vert}{{R}_{healthy} / {N}_{L}} ⌋ , \qquad ( 1 )$$

where L corresponds to the subject's current health level, Φ_{measured} corresponds to the actual (measured) value of the respective observable (e.g., weight, blood pressure, calorie intake, physical activity, body-mass-index, etc.), Φ_{healthy} corresponds to the healthy (i.e., target) value of the respective observable (e.g., weight, blood pressure, calorie intake, physical activity, body-mass-index, etc.) at the time of measurement, R_{healthy} corresponds to the healthy range of the respective observable, i.e., the range of values of the observable, where the subject is still considered healthy, and N_{L} corresponds to the number of health levels.

For instance, consider an example where the respective observable corresponds to the subject's Body Mass Index BMI, where the healthy range extends between 20 to 25 kg/m² (i.e., R_{healthy}=5), the target healthy value Φ_{healthy} is 22.5 kg/m² and the desired number of health levels N_{L} is 10, where the health levels are distributed symmetrically around the mean.

For a subject having a BMI of 24.55 kg/m², the subject's current health level is then calculated as follows:

$$L = ⌊ \frac{\vert {Φ}_{measured} - {Φ}_{healthy} \vert}{{R}_{healthy} / {N}_{L}} ⌋ = ⌊ \frac{\vert 24.55 - 22.5 \vert}{5 / 10} ⌋ = ⌊ 4.1 ⌋ = 4. \qquad ( 2 )$$

Accordingly, for a subject having a BMI of 21.9 kg/m², the subject's current health level yields:

$$L = ⌊ \frac{\vert {Φ}_{measured} - {Φ}_{healthy} \vert}{{R}_{healthy} / {N}_{L}} ⌋ = ⌊ \frac{\vert 21.9 - 22.5 \vert}{5 / 10} ⌋ = ⌊ 1.2 ⌋ = 1. \qquad ( 3 )$$

Optionally, the algorithm may take into account the type of physiological observable and whether the same distance below the average and above the average corresponds to an equally (un)healthy lifestyle. If not, the calculation may need to be weighted to reflect this health asymmetry around the mean. Optionally, the algorithm may further take into account the user's personal profile, such as age. For example, a low blood pressure with risk of fainting may be more of a health risk for an elderly frail person than for a young person between 20 and 30 years.

A detailed embodiment of the present invention may involve a definition of a mathematical model for the target value of a health parameter, a definition of bands parallel to the target value, and a definition of minimum and maximum values, indicating the outer bounds of the acceptable, healthy range. For a given subject the rate of change of the physiological observable under consideration is then calculated. One option to determine the degree of urgency is to consult a table defining the urgency of the feedback based on the difference between the measured and target value as well as the rate of change of the signal.

Optionally, each specific piece of health advice (e.g. eating less salt in case of high blood pressure; encourage walking in case of weight management; don't drink coffee before sleep in case of sleeping disorders etc.) is worded by a script writer for all the different levels of urgency and entered into a look-up table, as shown in the following table:

| | | message (“reduce salt intake”) |
|---|---|---|
| urgency | level 0 | “Did you know too salty food can cause high |
| level | | blood pressure?” |
| | level 1 | “Keep an eye on your salt intake.” |
| | level 2 | “Do remember to limit your salt intake.” |
| | level 3 | “Hey, you are being careful with your salt intake, |
| | | aren't you?” |
| | level 4 | “I urge you to really, really watch your salt intake.” |
| | level 5 | “Don't add extra salt, just don't.” |

Finally, depending on the level of urgency, the appropriate wording for the advice is picked from the look-up table.

As a further optional enhancement, different coaching styles may translate in urgency in different ways to address users with different psychological profiles. For instance, urgency may be translated into a tough, “drill sergeant” type feedback for performance-oriented consumers but can also be translated into different levels of pleading and encouragement for those receptive to a softer style of coaching.

In accordance with the present invention, different measurements and rates of change result in the same advice being offered in different tones of voice.

An example application of the invention refers generally to behavior change in all personal health related areas, including: weight management, stress management, sleep coaching, food intake, physical exercise, healthy pregnancy, and/or rehab (e.g. smoking, drinking, and drugs).

The physiological observable providing unit may comprise one or more input units and/or one or more sensors. Rather than determining an effectiveness rating based on a correlation of the assigned degree of urgency over time with the determined health level over time, the system may also provide the assigned degree of urgency over time and the determined health level over time to a further processing unit, which then performs the operative steps of the effectiveness analyzing unit.

Other variations to the disclosed embodiments can be understood and effected by those skilled in the art in practicing the claimed invention, from a study of the drawings, the disclosure, and the appended claims.

In the claims, the word “comprising” does not exclude other elements or steps, and the indefinite article “a” or “an” does not exclude a plurality.

A single unit or device may fulfill the functions of several items recited in the claims. The mere fact that certain measures are recited in mutually different dependent claims does not indicate that a combination of these measures cannot be used to advantage.

Determinations like determining a health level of the subject, determining a physiological observable of the subject, assigning a degree of urgency to the health advice, et cetera performed by one or several units or devices can be performed by any other number of units or devices. The determinations and/or the control of the system for providing a health advice to a subject in accordance with the above described method for providing a health advice to a subject can be implemented as program code means of a computer program and/or as dedicated hardware.

The health advice providing unit, the physiological observable providing unit, the rate of change providing unit, and the degree of urgency providing unit can be one single unit. Alternatively, the health advice providing unit, the physiological observable providing unit, the rate of change providing unit, and the degree of urgency providing unit are separate units. It shall be understood by the person skilled in the art that it is also possible to provide a single unit which provides the functionality of only some (e.g., of only two) of these units, where the remaining unit(s) are separate therefrom or respectively combined with each other.

A computer program may be stored/distributed on a suitable medium, such as an optical storage medium or a solid-state medium, supplied together with or as part of other hardware, but may also be distributed in other forms, such as via the Internet or other wired or wireless telecommunication systems. The term “computer program” may also refer to embedded software.

Any reference signs in the claims should not be construed as limiting the scope.

The present invention relates to a system, a device, a method, and a computer program for providing a health advice to a subject. It is proposed to modify a digital coaching advice depending on trends of a physiological observable (e.g., weight, Body Mass Index, etc.). A digital coach varies the wording of his/her advice depending on the risk categorization. It is proposed to analyze physiological measurements for deviation from the mean and rate of change and to translate these basic statistics into a concept of urgency/risk to structurally vary the wording or appearance of a digital coach. Thus, a digital coach as proposed herein may look, act and talk differently depending on the urgency/risk of the users. Coaches may vary in language and appearance from gentle, via hard-but-fair, to “drill sergeant”.

1. A system for providing a health advice to a subject, the system comprising:
a health level determining unit that is configured to determine a health level of the subject for a first time, wherein the health level is based on a comparison of a physiological observable of the subject at the first time to a target physiological observable of the subject;
a health advice providing unit that is configured to provide a health advice based on the physiological observable of the subject at the first time and on a rate of change of the physiological observable of the subject over time; and
an urgency assigning unit that is configured to assign a degree of urgency to the health advice based on the health level of the subject at the first time and on a rate of change of the physiological observable of the subject over time.

2. The system of claim 1,
wherein the system further comprises a user interface, wherein the system is configured provide an appearance of the user interface based on the degree of urgency.

3. The system of claim 1,
wherein the system is configured to provide a feedback to the user based on the assigned degree of urgency, wherein the feedback to the user is at least one of a visual feedback, an auditory feedback, a haptic feedback, and/or an olfactory feedback.

4. The system of claim 3,
wherein the at least one of a visual feedback, an auditory feedback, a haptic feedback, and/or an olfactory feedback based on the assigned degree of urgency are selected from a memory unit.

5. The system of claim 1,
wherein the system further comprises a physiological observable providing unit that is configured to provide the physiological observable of the subject at the first time.

6. The system of claim 5,
wherein the physiological observable providing unit comprises an input unit that is configured to receive the physiological observable of the subject at the first time; and/or wherein the physiological observable providing unit comprises a sensor that is configured to measure the physiological observable of the subject at the first time.

7. The system of claim 1,
wherein the system further comprises a feedback urgency database that is configured to store a table defining the degree of urgency depending on the health level of the subject at a given time and on the rate of change of the physiological observable of the subject over time.

8. The system of claim 1,
wherein the target physiological observable of the subject is dependent on time.

9. The system of claim 1,
wherein the system further comprises an effectiveness analyzing unit that is configured to correlate the assigned degree of urgency over time with the determined health level over time, wherein the effectiveness analyzing unit is further configured to provide an effectiveness rating based on the correlation.

10. The system of claim 1, wherein the system further comprises a display unit that is configured to display the health advice taking into account the assigned degree of urgency.

11. The system of claim 1, wherein the system further comprises a speaker unit that is configured to provide the health advice as an acoustic sound taking into account the assigned degree of urgency.

12. A device for providing a health advice to a subject, the device comprising:
a health advice providing unit that is configured to provide a health advice based on a physiological observable of the subject at a first time and on a rate of change of the physiological observable of the subject over time; and
a degree of urgency providing unit that is configured to provide a degree of urgency corresponding to the health advice based on the health level of the subject at the first time and on a rate of change of the physiological observable of the subject over time; and
a user interface that is configured to provide the health advice to the subject, wherein the user interface is configured to vary an appearance of the health advice depending on the degree of urgency.

13. The device of claim 12, wherein the user interface is configured to provide the health advice to the subject by means of at least one of a visual feedback, an auditory feedback, a haptic feedback, and/or an olfactory feedback;
wherein the user interface is configured to vary the at least one of a visual feedback, an auditory feedback, a haptic feedback, and/or an olfactory feedback depending on the degree of urgency.

14. A method for providing a health advice to a subject, the method comprising the steps of:
determining a health level of the subject for a first time, wherein the health level is based on a comparison of a physiological observable of the subject at the first time to a target physiological observable of the subject;
providing a health advice based on the physiological observable of the subject at the first time and on a rate of change of the physiological observable of the subject over time; and
assigning a degree of urgency to the health advice based on the health level of the subject at the first time and on a rate of change of the physiological observable of the subject over time.

15. A computer program for providing a health advice to a subject, the computer program comprising program code means for causing the system to carry out the steps of the method for providing a health advice to a subject as defined in claim 14, when the computer program is run on a computer controlling the system for providing a health advice to a subject.
